(19) United States
(12) Patent Application Publication
(10) Pub. No.: US 2026/0306673 A1
(43) Pub. Date: Oct. 8, 2026

(54) MODIFIED ADENO-ASSOCIATED VIRUS VECTOR AND USE THEREOF IN TREATING NEUROLOGICAL DISEASES

(71) Applicants: Bingsheng CHANG, Shanghai (CN); GENECOMBIO LTD., Shanghai (CN)

(72) Inventors: Ao ZHANG, Shanghai (CN); Zhouteng TAO, Shanghai (CN); Bingsheng CHANG, Shanghai (CN); Guirong DING, Shanghai (CN)

(21) Appl. No.: 19/571,113

(22) Filed: Mar. 18, 2026

Related U.S. Application Data
(63) Continuation of application No. PCT/CN2024/092657, filed on May 11, 2024.

(30) Foreign Application Priority Data
Sep. 18, 2023 (CN) 202311202549.4

(51) Int. Cl. A61K 48/00; A61K 9/00; A61P 25/16; C12N 9/88; C12N 15/113; C12N 15/86

(52) U.S. Cl. CPC A61K 48/005; A61K 9/0019; A61K 48/0075; A61P 25/16; C12N 9/88; C12N 15/1137; C12N 15/86; C12N 2310/141; C12N 2750/14143; C12N 2830/15; C12N 2830/50; C12Y 401/01028

(57) ABSTRACT

Provided in the present application is a nucleic acid fragment which comprises a modified nucleotide sequence encoding an aromatic L-amino acid decarboxylase (AADC). Further provided in the present application are an expression vector comprising the nucleic acid fragment, a recombinant adeno-associated virus (rAAV) comprising the expression vector, a pharmaceutical composition comprising the above-mentioned nucleic acid fragment, expression vector, or rAAV, and the use thereof in treating neurological diseases.

### CROSS-REFERENCE TO RELATED APPLICATION

This application is a continuation of PCT Application No. PCT/CN2024/092657, filed on May 11, 2024, which is based upon and claims priority to Chinese Patent Application No. 202311202549.4 filed on Sep. 18, 2023, all of which are incorporated by reference in their entireties for all purposes.

### REFERENCE TO AN ELECTRONIC SEQUENCE LISTING

The content of the electronic sequence listing (D-CF230022US_Sequence Listing.xml; Size: 100,528 bytes; and Date of Creation: Apr. 20, 2026) is incorporated by reference in its entirety.

### TECHNICAL FIELD

The present application relates to the field of biomedicine, specifically to a nucleic acid fragment comprising a nucleotide sequence encoding an aromatic L-amino acid decarboxylase, an expression vector, and use thereof in the treatment of a neurological disease.

### BACKGROUND

Aromatic L-amino acid decarboxylase (AADC) is a homodimeric pyridoxal phosphate-dependent enzyme responsible for the synthesis of dopamine and serotonin. AADC catalyzes the decarboxylation of L-3,4-dihydroxyphenylalanine (L-DOPA or levodopa) to dopamine, the decarboxylation of L-5-hydroxytryptophan (L-5-HTP) to serotonin, and the decarboxylation of L-tryptophan to tryptamine.

Aromatic L-amino acid decarboxylase deficiency (AADCD) is an autosomal recessive inherited neurometabolic disorder caused by defects in the gene, resulting in AADC deficiency, which in turn affects the synthesis of dopamine and serotonin from L-DOPA and L-5-HTP, respectively. In the absence of neuronal dopamine, patients may experience dyskinesia, behavioral problems, autonomic dysfunction, and developmental delays. Traditional pharmacotherapy for AADC deficiency is a combination of vitamin B6, dopamine agonists, and monoamine oxidase inhibitors, with limited efficacy. A promising therapeutic approach for aromatic L-amino acid decarboxylase deficiency is AADC gene therapy.

Parkinson's disease (PD) is a progressive neurodegenerative disease of the central nervous system (CNS), characterized by the gradual loss of dopaminergic neurons, leading to dopamine deficiency. Exogenous L-DOPA supplementation is currently one of the most effective strategy for the treatment of Parkinson's disease. However, as PD progresses, AADC levels gradually decrease, necessitating an increase in L-DOPA dose to maintain the desired clinical efficacy. Additionally, the benefit of dopamine therapy becomes less marked over time, partly due to the progressive death of dopamine-generating cells and the corresponding loss of AADC activity. Furthermore, systemic administration of high-dose dopamine is complicated by side effects, such as fluctuations in motor performance, dyskinesia, and hallucinations, resulting from dopaminergic stimulation of the mesolimbic system. Accordingly, one strategy to restore dopaminergic function and minimize side effects is to deliver AADC directly to a targeted region of the CNS using gene therapy (EMBO Mol Med., 2021 Sep. 7, 13(9):e14712; CN202011500265.X).

Adeno-associated virus (AAV), a helper virus-dependent DNA parvovirus belonging to the genus Dependovirus, is an attractive gene therapy vector due to its long-term gene expression, inability to autonomously replicate without a helper virus, ability to transduce dividing and non-dividing cells, and lack of pathogenicity from wild-type infections (Mol. Ther., 2010, 18(8):1458-1461). However, existing AAV vectors used in gene therapy have certain limitations. For example, the expression efficiency of the gene of interest is low; the AAV capsid, depending on different serotypes, may elicit immunogenicity, thus leading to safety issues; and the gene of interest may be overexpressed in non-target organs, thus resulting in toxic side effects. It is evident that there remains a need for AAV research and development in the field of gene therapy, and existing AAV vectors require further improvement.

It is to be noted that the methods described in this section are not necessarily those that were previously conceived or adopted. Unless otherwise specified, no method described in this section shall be construed as prior art simply by virtue of its inclusion in this section. Similarly, unless otherwise specified, the issues mentioned in this section shall not be construed as being admitted in any prior art.

### SUMMARY

To address the aforementioned technical issues, the present application provides a nucleic acid fragment comprising a nucleotide sequence encoding AADC, wherein the nucleotide sequence encoding AADC comprises a nucleotide sequence having at least 80%, 85%, 90%, 95%, 98%, 99%, or 100% identity to the nucleotide sequence shown in SEQ ID NO: 3. The present application effectively improves the expression efficiency by subjecting the nucleotide sequence encoding AADC to codon optimization.

According to an embodiment of the present application, there is further provided an expression vector comprising the nucleic acid fragment described herein. In addition to codon optimization of the nucleotide sequence encoding AADC, the expression vector herein further incorporates promoter and enhancer elements with high expression efficiency to further increase the expression level of AADC, thereby achieving an effective therapeutic effect.

In some embodiments, the expression vector further comprises miRNA target sequences.

By leveraging the ability of miRNAs to inhibit gene expression at specific sites, the expression of expression vectors in non-target organs can be reduced, thereby mitigating the potential toxicity of gene therapy.

According to an embodiment of the present application, there is further provided a recombinant adeno-associated virus (rAAV) comprising the expression vector described herein.

According to an embodiment of the present application, there is further provided a pharmaceutical composition comprising the nucleic acid fragment described herein, the expression vector described herein, and/or recombinant adeno-associated virus described herein, as well as pharmaceutically, physiologically, or nutritionally acceptable excipients.

According to an embodiment of the present application, there is further provided use of the nucleic acid fragment described herein, the expression vector described herein, the recombinant adeno-associated virus described herein, or the pharmaceutical composition described herein in the preparation of a medicament for preventing, alleviating, or treating a neurological disease.

According to an embodiment of the present application, there is further provided a method for preventing, alleviating, or treating a neurological disease, comprising administering to a subject in need thereof a therapeutically effective amount of the nucleic acid fragment described herein, the expression vector described herein, the recombinant adeno-associated virus described herein, or the pharmaceutical composition described herein.

It is to be understood that the content described in this section is not intended to identify key or important features of the embodiments of the present application, nor is it intended to limit the scope of the present application. Other features of the present application will become readily apparent from the following description.

### BRIEF DESCRIPTION OF DRAWINGS

The drawings exemplarily illustrate embodiments, form a part of the description, and serve to explain exemplary implementations of the embodiments together with the texts of the description. The embodiments shown are for illustrative purposes only and do not limit the scope of the claims. Throughout all the drawings, identical reference numbers designate similar, but not necessarily identical, elements.

FIG. 1 illustrates a schematic diagram of the structure of the pAADC_v0 vector prepared in Embodiment 1.

FIG. 2 illustrates a schematic diagram of the structure of the pAADC_v1 vector prepared in Embodiment 1.

FIG. 3 illustrates a schematic diagram of the structure of the pAADC_v2 vector prepared in Embodiment 1.

FIG. 4 illustrates a schematic diagram of the structure of the pAADC_vA vector prepared in Embodiment 1.

FIG. 5 illustrates a schematic diagram of the structure of the pAADC_vB vector prepared in Embodiment 1.

FIG. 6 illustrates a schematic diagram of the structure of the pAADC_vC vector prepared in Embodiment 1.

FIG. 7 illustrates a schematic diagram of the structure of the pAADC_vE vector prepared in Embodiment 1.

FIG. 8 illustrates a schematic diagram of the structure of the pAADC_vF vector prepared in Embodiment 1.

FIG. 9 illustrates the Western blot results representing the effects of pAADC_v0, pAADC_v1, and pAADC_v2 on the expression of AADC in 293T cells in Embodiment 2.

FIG. 10 illustrates the Western blot results representing the effects of pAADC_vA and pAADC_vB on the expression of AADC in 293T cells in Embodiment 3.

FIG. 11 illustrates the Western blot results representing the effects of pAADC_v1 and pAADC_vC on the expression of AADC in 293T cells in Embodiment 4.

FIG. 12 illustrates the expression results of the protein of interest after transfection of Neuro-2a cells with different vectors in Embodiment 5. Different labels in the figure represent the use of different vectors for transfection: blank represents the untransfected blank group, PC represents the control vector not comprising miR-183 and miR-122 target sequences, 14 represents the vector comprising miR-183 and miR-122 target sequences, Let-7a-1 represents the vector comprising the let-7a-1 target sequence, 802 represents the vector comprising the miR-802 target sequence, 99a-5p represents the vector comprising the miR-99a-5p target sequence, 100-5p represents the vector comprising the miR-100-5p target sequence, and 1200 represents the vector comprising the miR-1200 target sequence.

FIG. 13 illustrates the Western blot results representing the effects of AAV-AADC_v1, AAV-AADC_vE, and AAV-AADC_vF on the expression of AADC in 293T cells in Embodiment 6.

FIG. 14 illustrates the Western blot results representing the effects of AAV9-AADC_vA, AAV9-AADC_v1, AAV1-AADC_v1, and AAV2-AADC_v1 on the expression of AADC in mouse brain tissue in Embodiment 7.

FIG. 15 illustrates the immunofluorescence staining result representing the effect of AAV9-AADC_vA on the expression of AADC in mouse brain tissue in Embodiment 7.

FIG. 16A illustrates the result representing the effect of AAV2-hAADC (the AADC expression vector in the prior art) on the expression of AADC in rat brain tissue in Embodiment 8. FIG. 16B illustrates the DAB staining result representing the effect of AAV9-AADC_vA on the expression of AADC in rat brain tissue in Embodiment 8.

FIG. 17 illustrates the Western blot results and statistical graphs representing the effects of AAV9-AADC_vE and AAV9-AADC_vA on the expression of AADC in mouse brain and liver tissues in Embodiment 9.

FIG. 18 illustrates the behavioral test results of the Parkinson's rat model before drug treatment in Embodiment 10. ns represents no statistically significant difference.

FIG. 19 illustrates the statistical results representing the effect of AAV9-AADC_vA on the behaviors of a Parkinson's rat model in Embodiment 10. Group 1 is the test results of rats injected with empty capsids; Group 2 is the test results of rats injected with virus AAV9-AADC_vA. * represents statistically significant difference, and ns represents no statistically significant difference.

FIG. 20 illustrates the statistical result representing the effect of AAV9-AADC_vA on the behavior of a Parkinson's rat model in Embodiment 10. Group 1 is the test results of rats injected with empty capsids; Group 2 is the test results of rats injected with the virus AAV9-AADC_vA. * and ** represent statistically significant difference.

FIG. 21 illustrates the test result of striatal dopamine content in a Parkinson's rat model using AAV9-AADC_vA in Embodiment 11. Group 1 is the test results of rats injected with empty capsids; Group 2 is the test results of rats injected with the virus AAV9-AADC_vA. ** represents statistically significant difference.

### DETAILED DESCRIPTION

Unless otherwise specified, all figures used in the description and claims to indicate content, concentration, proportion, mass, volume, time, temperature, thickness, technical effect, etc., shall in any case be understood to be modified by the terms “about” or “approximately”. Accordingly, unless otherwise indicated, the numerical parameters listed in the following description and appended claims are approximate values. For those skilled in the art, they may vary depending on the desired nature and effect sought to be obtained through the present application, and each numerical parameter shall be interpreted according to the number of significant figures and conventional rounding methods or in a manner understood by those skilled in the art.

Although the numerical ranges and parameters described in the present application are approximate, the numerical values described in the specific embodiments are provided as precisely as possible. However, any numerical value will inherently comprise some errors, which are necessarily caused by the standard deviation found in its corresponding test measurement. Each range of values given in this description will comprise every narrower range of values that falls within the wider range, just as these narrower ranges are explicitly stated herein.

Unless otherwise specified or contradicted by the context, the terms or expressions used herein are to be read in the context of the whole document and as understood by one of ordinary skill in the art. Unless otherwise specified, all technical and scientific terms used herein have the same meaning as commonly understood by one of ordinary skill in the art.

When used herein, the expression “A and/or B” comprises three cases: (1) A; (2) B; and (3) A and B. The expression “A, B, and/or C” comprises seven cases: (1) A; (2) B; (3) C; (4) A and B; (5) A and C; (6) B and C; and (7) A, B, and C. The meaning of similar expressions can be inferred in the same way.

When used herein, “nucleic acid” and “polynucleotide” are used interchangeably, referring to a polymer of nucleotides of any length, and comprising deoxyribonucleotides, ribonucleotides, combinations thereof and analogues.

When used herein, “polypeptide” and “peptide” are used interchangeably, referring to an amino acid polymer of any length. Accordingly, polypeptides, oligopeptides, proteins, antibodies, and enzymes are all comprised in the definition of polypeptides.

It is to be noted that, in the context of the present application, upstream region refers to the 5′ end of a gene or the N-terminus of a protein, and downstream region refers to the 3′ end of a gene or the C-terminus of a protein, with the direction from the upstream region to the downstream region being from the 5′ end to the 3′ end or from the N-terminus to the C-terminus.

When used herein, “vector” refers to a nucleic acid molecule that can transport another nucleic acid molecule linked to it. Examples of vectors comprise, but are not limited to, plasmids, viruses, bacteria, bacteriophages, and insertable DNA fragments.

The “adeno-associated virus vector” described herein is derived from adeno-associated virus. Adeno-associated virus vectors are derived from the wild-type genome of the virus by removing all or part of the wild-type genome from the adeno-associated virus genome using molecular methods and replacing it with heterologous (non-natural) nucleic acids (such as nucleic acids encoding therapeutic proteins or polynucleotide sequences). Typically, for an adeno-associated virus vector, one or two inverted terminal repeat (ITR) sequences from the adeno-associated virus genome are retained within the adeno-associated virus vector. Adeno-associated virus vectors can be used as gene therapy vectors because they can introduce nucleic acids/genetic material into cells, allowing the nucleic acids/genetic material to remain in the cells.

The term “serotype” described herein refers to an adeno-associated virus that has a capsid that is serologically different from other adeno-associated virus serotypes. Serological specificity is determined based on the lack of cross-reactivity between antibodies to one adeno-associated virus compared to another adeno-associated virus. Such cross-reactivity differences are typically due to differences in capsid protein sequences/antigenic determinants (e.g., due to differences in VP1, VP2, and/or VP3 sequences of adeno-associated virus serotypes). Under the traditional definition, a serotype refers to a virus of interest that has been tested for neutralizing activity against sera that are specific to all existing and characterized serotypes, and for which no antibodies have been found to neutralize the virus of interest. As more naturally occurring viral isolates are discovered and/or capsid mutants are generated, serological differences from any of the currently existing serotypes may or may not exist. Accordingly, in the absence of serological differences in a new virus (e.g., adeno-associated virus), such a new virus (e.g., adeno-associated virus) will be a subgroup or variant of the corresponding serotype. In many cases, serological tests for neutralizing activity have not been performed on mutant viruses with capsid sequence modifications to determine whether they are another serotype according to the traditional serotype definition. Accordingly, for convenience and to avoid repetition, the term “serotype” is used broadly to refer to serologically different viruses (e.g., adeno-associated virus) as well as viruses that are not serologically different but may be within a subgroup or variant of a given serotype (e.g., adeno-associated virus).

When used herein, “miRNA target sequence” and “miRNA binding site” are used interchangeably, referring to the nucleotide sequence that can bind to a specific miRNA. In some non-limiting embodiments of the present application, the miRNA target sequences are designed at the 3′ end of the AADC gene, which can bind to specific miRNAs to inhibit the expression level of the AADC gene.

When used herein, “operable linkage” refers to the linkage among multiple nucleic acid fragments in a functional relationship. When one nucleic acid forms a functional relationship with another nucleic acid sequence, it is “operably linked”. For example, if a promoter or other transcriptional regulatory sequence affects the transcription of a coding sequence (or gene), it can be operably linked to the coding sequence in a manner that the promoter can induce the transcription of the gene. Operably linked means that the nucleotide sequences being ligated may be contiguous or noncontiguous.

When used herein, the terms “alleviation”, “treatment”, and their synonyms refer to the improvement of a disease, symptom, and/or condition. “Alleviation” and “treatment” can be an improvement in at least one measurable physical parameter, which may not necessarily be identifiable by the patient. “Alleviation” and “treatment” can also be the physical (e.g., stabilizing identifiable symptoms), physiological (e.g., stabilizing physical parameters), or both, suppression of the progression of a disease, symptom, and/or condition. “Alleviation” and “treatment” can also be the act of slowing the progression of a disease, symptom, and/or condition, or reversing it.

When used herein, the term “prevention” and its synonyms refer to the act of delaying the onset of a particular disease, symptom and/or condition or symptoms associated with such disease, symptom and/or condition, or reducing the risk of acquiring such disease, symptom and/or condition.

The term “pharmaceutically acceptable excipient” described herein refers to a carrier, diluent, or adjuvant used in the preparation or administration of a drug, which is not an essential active ingredient and does not have excessive toxicity after administration. Suitable pharmaceutically acceptable excipients are well known to one of ordinary skill in the art, comprising, but not limited to, calcium bicarbonate, calcium phosphate, various sugar and starch types, cellulose derivatives, gelatin, vegetable oils, polyethylene glycol, and surfactants, comprising, for example, polysorbate 20.

The terms “physiologically acceptable excipient” and “nutritionally acceptable excipient” described herein refer to carriers, diluents, or adjuvants that do not cause significant irritation to organisms and do not eliminate the biological activity and properties of the applied polypeptide.

To make the above-mentioned objectives, features and advantages of the present application more apparent and understandable, the specific embodiments of the present application are described in detail below.

### Nucleic Acid Fragment, Expression Vector, Recombinant Adeno-Associated Virus

According to an embodiment of the present application, provided is a nucleic acid fragment comprising a nucleotide sequence encoding AADC, wherein the nucleotide sequence encoding AADC comprises a nucleotide sequence having at least 80%, 85%, 90%, 95%, 98%, 99%, or 100% identity to the nucleotide sequence shown in SEQ ID NO: 3. In some embodiments, the nucleotide sequence encoding AADC comprises the nucleotide sequence shown in SEQ ID NO: 3. In some embodiments, the nucleotide sequence encoding AADC is the nucleotide sequence shown in SEQ ID NO: 3.

According to an embodiment of the present application, there is further provided an expression vector comprising the nucleic acid fragment described herein.

In some embodiments, the expression vector further comprises miRNA target sequences. miRNA (microRNA) is a single-stranded non-coding RNA with 18 to 25 nucleotides in length that is widely distributed in humans and animals. The RNA-induced silencing complex formed by miRNA and protein factors can recognize target sequences in mRNA, reduce the mRNA expression level by degrading mRNA molecules, promoting 3′ end deadenylation of mRNA molecules, and inhibiting translation, thereby regulating gene expression at the posttranscriptional level (Nat Rev Mol Cell Biol., 2005, 6(5): 376-385). By using an miRNA that is highly expressed in a certain cell, inserting the target sequence of the miRNA into the 3′UTR of a foreign gene can effectively inhibit the expression of the foreign gene in that cell. In some preferred embodiments, the miRNA target sequences can reduce the expression efficiency of the nucleotide sequence encoding AADC in liver tissues. In some preferred embodiments, the miRNA target sequences can reduce the expression efficiency of the nucleotide sequence encoding AADC in dorsal root ganglion tissues. In some preferred embodiments, the miRNA target sequences are selected from at least one or more of miR-183, miR-182, miR-96, miR-23b, miR-145, miR-148a, miR-22, miR-122, miR-143, miR-21, and miR-192. In some preferred embodiments, the miRNA target sequences comprise one or both of the miR-183 target sequence and the miR-122 target sequence. In some preferred embodiments, the miRNA sequence comprises one or both of the nucleotide sequences shown in SEQ ID NO: 5 or SEQ ID NO: 6. In some embodiments, the number of miRNA target sequences is 1, 2, 3, 4, 5, 6, 7, or 8. In some preferred embodiments, the number of miRNA target sequences is 4. In some preferred embodiments, the miRNA target sequences comprise 2 miR-183 target sequences and 2 miR-122 target sequences. The more than one miRNA target sequences can be linked together in various ways known in the art. In some preferred embodiments, the more than one miRNA target sequences are operably linked by a spacer sequence comprising the nucleotide sequence shown in any one of SEQ ID NOs: 16 to 18. In some preferred embodiments, the more than one miRNA target sequences are operably linked in a cross-arranged and mutually spaced apart way.

In some embodiments, the expression vector further comprises a promoter. The promoter can be any suitable promoter sequence, that is, a nucleic acid sequence that can be recognized by the host cell expressing the nucleic acid sequence. The promoter sequence comprises transcriptional regulatory sequences that mediate the expression of proteins or peptides. A promoter can be any nucleic acid sequence that is transcriptionally active in the selected host cell, comprises mutant, truncated, and hybrid promoters, and can be derived from genes that encode extracellular or intracellular proteins or polypeptides that are homologous or heterologous to those of the host cell. In some embodiments, the promoter comprises a chicken beta actin (CBA) promoter, a cytomegalovirus (CMV) promoter, and a human SYN1 gene promoter.

In some embodiments, the expression vector may optionally further comprise an enhancer. The enhancer can be any suitable enhancer sequence. In some embodiments, the enhancer is a CMV enhancer.

In some embodiments, the expression vector further comprises a polyadenylation signal. Transcription termination sequences known in the art can be used in the present application. In some embodiments, the polyadenylation signal is a bovine growth hormone (bGH) polyadenylation signal or a simian vacuolating virus 40 (SV40) polyadenylation signal.

In some embodiments, the expression vector comprises, in sequence from the 5′ end to the 3′ end: a 5′ end adeno-associated virus (AAV) ITR sequence; a promoter; the nucleic acid fragment described herein; a polyadenylation signal; and a 3′ end AAV ITR sequence.

In some embodiments, the expression vector comprises, in sequence from the 5′ end to the 3′ end: a 5′ end AAV ITR sequence; a promoter; the nucleic acid fragment described herein; miRNA target sequences; a polyadenylation signal; and a 3′ end AAV ITR sequence.

In some embodiments, the expression vector comprises, in sequence from the 5′ end to the 3′ end: a 5′ end AAV ITR sequence; an enhancer; a promoter; the nucleic acid fragment described herein; a polyadenylation signal; and a 3′ end AAV ITR sequence.

In some preferred embodiments, the expression vector comprises, in sequence from the 5′ end to the 3′ end: a 5′ end AAV ITR sequence; an enhancer; a promoter; the nucleic acid fragment described herein; miRNA target sequences; a polyadenylation signal; and a 3′ end AAV ITR sequence.

The expression vector can be delivered using any suitable vector. In some embodiments, the expression vector is a plasmid vector. In some embodiments, the expression vector is a viral vector. Suitable plasmid vectors and viral vectors are well known in the art.

Viral particles comprising the gene expression cassette described herein are typically generated in packaging cells that are capable of replicating the viral genome, expressing viral proteins, and assembling viral particles. The technique for generating AAV vector particles in packaging cells is well known in the art. In some non-limiting embodiments, packaging cells can be generated by simply transforming suitable cells with one or more plasmids encoding the AAV genome, AAV proteins, and any desired helper virus functions, a method known as “triple transfection” using three plasmids, each carrying a set of such genes. See Grieger et al., Nature Protocols, 2006, 1(3): 1412-28.

In some preferred embodiments, the expression vector is an AAV vector. In some embodiments, the expression vector further comprises a 5′ end AAV ITR sequence and a 3′ end AAV ITR sequence. In some preferred embodiments, the 5′ end AAV ITR sequence is the nucleotide sequence shown in SEQ ID NO: 12 or SEQ ID NO: 14, and the 3′ end AAV ITR sequence is the nucleotide sequence shown in SEQ ID NO: 13 or SEQ ID NO: 39. Compared with the wild-type 5′ end AAV ITR sequence (SEQ ID NO: 14), the mutant 5′ end AAV ITR sequence (SEQ ID NO: 12) can further enhance the expression efficiency of the gene of interest.

According to an embodiment of the present application, there is further provided a recombinant adeno-associated virus (rAAV) comprising the expression vector described herein. In some embodiments, the rAAV serotypes comprise AAV1, AAV2, AAV3, AAV4, AAV5, AAV6, AAV7, AAV8, AAV9, AAV9.47, AAV9 (hu14), AAV10, AAV11, AAV12, AAV13, AAVrh8, AAVrh10, AAV-DJ, or AAV-DJ8. In some preferred embodiments, the rAAV serotypes comprise AAV1, AAV2, and AAV9. In some preferred embodiments, the rAAV serotype is AAV9. The nucleotide sequences of the genomes of different AAV serotypes are known in the art.

### Pharmaceutical Composition

According to an embodiment of the present application, there is further provided a pharmaceutical composition comprising the nucleic acid fragment described herein, the expression vector described herein, and/or recombinant adeno-associated virus described herein, as well as pharmaceutically, physiologically, or nutritionally acceptable excipients.

In some embodiments, the pharmaceutical composition of the present application may, in addition to the nucleic acid fragment, expression vector and/or recombinant adeno-associated virus provided herein, further comprises pharmaceutically, nutritionally or physiologically acceptable carriers, such as liquid, gel or solid carriers, aqueous vehicles, non-aqueous vehicles, antimicrobial agents, isotonic agents, buffering agents, antioxidants, suspending/dispersing agents, chelating agents, diluents, adjuvants, excipients or non-toxic auxiliary substances, other components known in the art or various combinations thereof.

In some embodiments, the pharmaceutical composition is administered by intravenous injection or suboccipital injection.

### Use

According to an embodiment of the present application, there is further provided use of the nucleic acid fragment described herein, the expression vector described herein, the recombinant adeno-associated virus described herein, or the pharmaceutical composition described herein in the preparation of a medicament for preventing, alleviating, or treating a neurological disease. In some embodiments, the neurological disease is a neurological disease caused by the deficiency or reduced activity of aromatic L-amino acid decarboxylases. In some embodiments, the neurological disease comprises Parkinson's disease and aromatic L-amino acid decarboxylase deficiency.

### Disease Treatment Method

According to an embodiment of the present application, there is further provided a method for preventing, alleviating, or treating a neurological disease, comprising administering to a subject in need thereof a therapeutically effective amount of the nucleic acid fragment described herein, the expression vector described herein, the recombinant adeno-associated virus described herein, or the pharmaceutical composition described herein. In some embodiments, the neurological disease is a neurological disease caused by the deficiency or reduced activity of aromatic L-amino acid decarboxylases. In some embodiments, the neurological disease comprises Parkinson's disease and aromatic L-amino acid decarboxylase deficiency.

The specific dosage and treatment regimen for any particular patient will depend on a variety of factors comprising the activity of the specific expression vector used, age, weight, general health condition, sex, diet, timing of administration, excretion rate, drug combination, severity and course of the disease, condition or symptom, the patient's predisposition to the disease, condition or symptom, and the judgment of the treating physician.

The effective amount can be administered in one or more administrations, applications or dosages. The expression vector or the pharmaceutical composition can be administered once or more per day to once or more per week; comprising once every other day. Those skilled in the art will understand that certain factors may affect the dosage and timing required to effectively treat a subject and comprise, but are not limited to, the severity of the disease or symptom, previous treatment, the overall health and/or age of the subject, and other diseases present. Furthermore, treating a subject with a therapeutically effective amount of the therapeutic compound described herein may comprise a single treatment or a series of treatments. For example, an effective amount can be administered at least once.

The various embodiments and preferences disclosed above can be combined with each other (as long as they are not inherently contradictory to each other), and the various embodiments formed by such combination are all considered as part of the disclosure of the present application.

### Embodiments

The following is a description of exemplary embodiments of the present application in conjunction with the drawings, comprising various details of the embodiments of the present application to facilitate understanding. It is to be understood that they are considered to be merely exemplary and are by no means intended to limit the scope of protection of the present application. The scope of protection of the present application is limited only by the claims. Accordingly, one of ordinary skill in the art is to recognize that various changes and modifications can be made to the embodiments described here without departing from the scope of the present application. Similarly, for the sake of clarity and conciseness, descriptions of well-known functions and structures are omitted in the following description.

Unless otherwise specified, the reagents and instruments used in the following embodiments are all commercially available conventional products. Unless otherwise specified, the experiments were performed under conventional conditions or conditions recommended by the manufacturer.

### Embodiment 1: Construction of Expression Vector

### 1.1 Nucleotide Sequence Encoding AADC

The amino acid sequence of human AADC protein (SEQ ID NO: 2; GenBank: NP_001076440.2) and the nucleotide sequence encoding human AADC (SEQ ID NO: 1; GenBank: NM_001082971.2) were searched through the NCBI GenBank database (https://www.ncbi.nlm.nih.gov/gene), and the AADC protein coding region sequence was subjected to codon optimization to obtain the AADC optimized sequence AZ3 (SEQ ID NO: 3) and the AADC optimized sequence EF (SEQ ID NO: 4).

### 1.2 Construction of Adeno-Associated Virus Vector

The adeno-associated virus vector constructed in this embodiment comprises, in sequence from the 5′ end to the 3′ end: a 5′ end ITR; an enhancer; a promoter; a regulatory sequence; a nucleotide sequence encoding human AADC; miRNA target sequences; a polyadenylation signal; a 3′ end AAV ITR sequence; and a backbone sequence.

TABLE 1
Sequence information of expression vector

| | | | Sequence Source |
|---|---|---|---|
| Structure | Name | Sequence No. | Reference |
| 5′ end ITR | 5′ end AAV ITR sequence | SEQ ID NO: 14 | NCBI GenBank ID |
| | | | (MN224160.1) |
| | Mutated 5′ end AAV ITR | SEQ ID NO: 12 | / |
| | sequence | | |
| Enhancer | CMV enhancer | SEQ ID NO: 8 | NCBI GenBank ID |
| | | | (MN072725.1) |
| Promoter | CMV promoter | SEQ ID NO: 7 | NCBI GenBank ID |
| | | | (MN072725.1) |
| | CBA promoter | / | NCBI GenBank ID |
| | | | (MK225672.1) |
| | Human SYN1 promoter | SEQ ID NO: 19 | / |
| Regulatory | hGlobin intro2 exon3 intron | SEQ ID NO: 10 | Patent (U.S. Pat. No. |
| sequence | regulatory sequence | | 10,898,585B2) |
| | Chimeric chβ-Actini-MVMj | SEQ ID NO: 20 | Literature (Hum Gene |
| | regulatory sequence | | Ther., 2011, 22(9): |
| | | | 1143-53.) |
| Nucleotide | Wild-type sequence | SEQ ID NO: 1 | NCBI GenBank ID |
| sequence | encoding AADC | | (NM_001082971.2) |
| encoding | Optimized sequence AZ3 | SEQ ID NO: 3 | / |
| human | Optimized sequence EF | SEQ ID NO: 4 | / |
| AADC | | | |
| miRNA | mir-183 target sequence | SEQ ID NO: 5 | / |
| target | mir-122 target sequence | SEQ ID NO: 6 | / |
| sequence | | | |
| Polyadenylation | Bovine growth hormone | SEQ ID NO: 11 | NCBI GenBank ID |
| signal | polyadenylation signal | | (OQ749646.1) |
| | Simian vacuolating virus 40 | SEQ ID NO: 9 | NCBI GenBank ID |
| | polyadenylation signal | | (OP902172.1) |
| 3′ end ITR | Mutated 3′ end AAV ITR | SEQ ID NO: 13 | NCBI GenBank ID |
| | sequence | | (MK163941.1) |
| | 3′ end AAV ITR Sequence | SEQ ID NO: 39 | / |
| Backbone | Plasmid backbone with | SEQ ID NO: 15 | NCBI GenBank ID |
| sequence | kanamycin resistance | | (MK225672.1, |
| | | | KY985380.1, |
| | | | MK225672.1) |

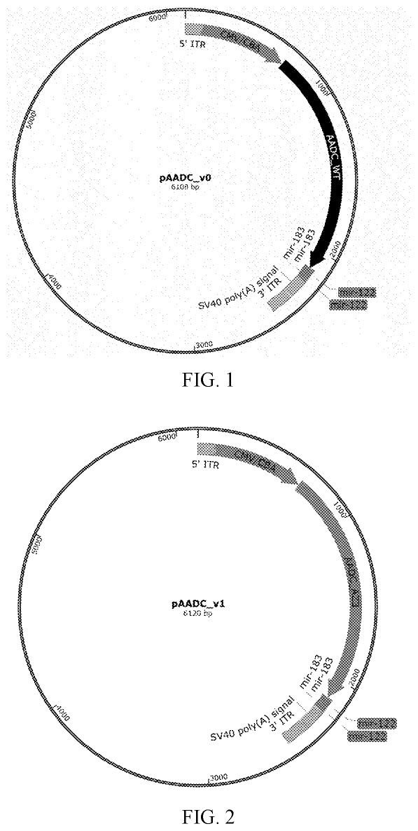

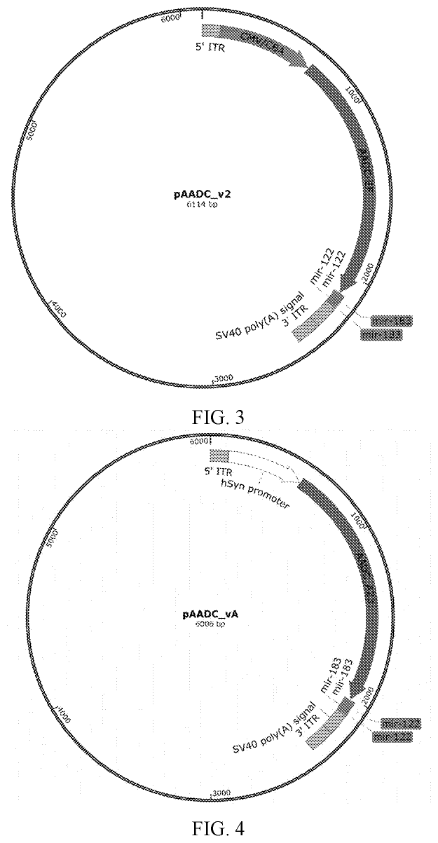

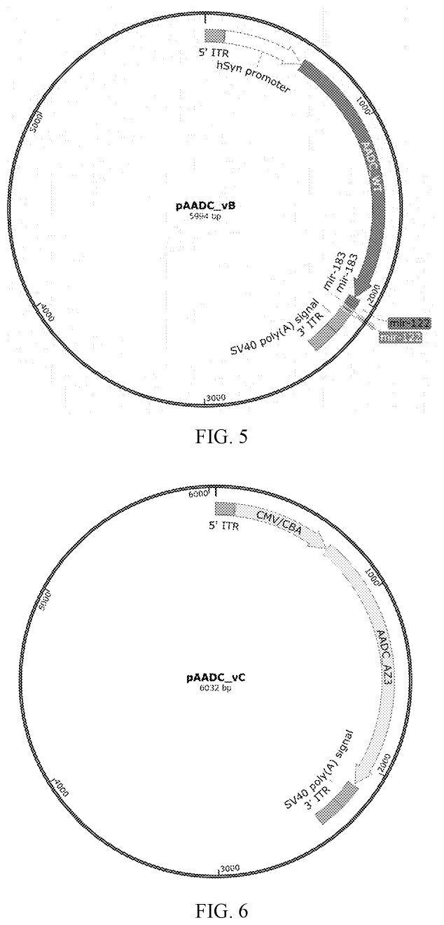

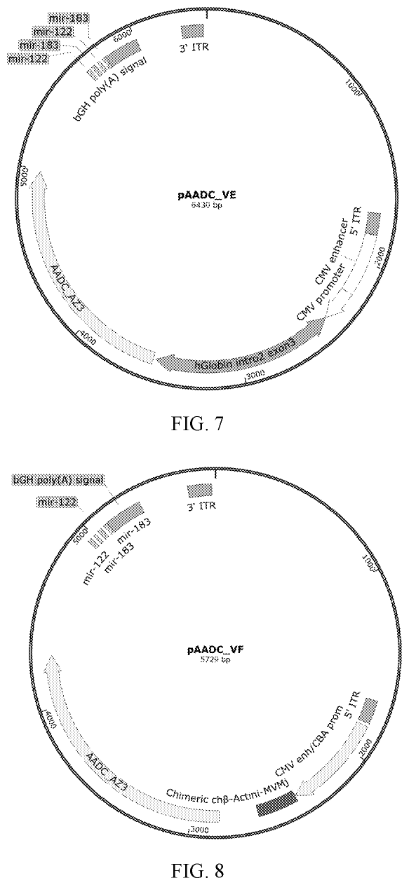

According to the sequence information in Table 1, the designed gene sequence was synthesized through gene synthesis services (GeneScript, Tsingke, etc.) to construct the following vectors:
pAADC_v0 has a structure as illustrated in FIG. 1, and comprises a mutated 5′ end AAV ITR sequence, a CMV enhancer, a CBA promoter, a wild-type sequence encoding AADC, mir-183 and mir-122 target sequences, a simian vacuolating virus 40 polyadenylation signal, a mutated 3′ end AAV ITR sequence, and a backbone sequence.
pAADC_v1 has a structure as illustrated in FIG. 2, and comprises a mutated 5′ end AAV ITR sequence, a CMV enhancer, a CBA promoter, the optimized sequence AZ3, mir-183 and mir-122 target sequences, a simian vacuolating virus 40 polyadenylation signal, a mutated 3′ end AAV ITR sequence, and a backbone sequence.
pAADC_v2 has a structure as illustrated in FIG. 3, and comprises a mutated 5′ end AAV ITR sequence, a CMV enhancer, a CBA promoter, the optimized sequence EF, mir-183 and mir-122 target sequences, a simian vacuolating virus 40 polyadenylation signal, a mutated 3′ end AAV ITR sequence, and a backbone sequence.
pAADC_vA has a structure as illustrated in FIG. 4, and comprises a mutated 5′ end AAV ITR sequence, a human SYN1 promoter, the optimized sequence AZ3, mir-183 and mir-122 target sequences, a simian vacuolating virus 40 polyadenylation signal, a mutated 3′ end AAV ITR sequence, and a backbone sequence.
pAADC_vB has a structure as illustrated in FIG. 5, and comprises a mutated 5′ end AAV ITR sequence, a human SYN1 promoter, a wild-type sequence encoding AADC, mir-183 and mir-122 target sequences, a simian vacuolating virus 40 polyadenylation signal, a mutated 3′ end AAV ITR sequence, and a backbone sequence.
pAADC_vC has a structure as illustrated in FIG. 6 which is basically the same as that of pAADC_v1, except that the pAADC_vC vector does not comprise mir-183 and mir-122 target sequences.
pAADC_vE has a structure as illustrated in FIG. 7, and comprises a 5′ end AAV ITR sequence, a CMV promoter, a CMV enhancer, the hGlobin intro2 exon3 intron regulatory sequence, the optimized sequence AZ3 encoding AADC, mir-183 and mir-122 target sequences, a bovine growth hormone (bGH) polyadenylation signal, a 3′ end AAV ITR sequence, and a backbone sequence.
pAADC_vF has a structure as illustrated in FIG. 8, and comprises a 5′ end AAV ITR sequence, a CMV enhancer, a CBA promoter, the Chimeric chβ-Actini-MVMj regulatory sequence, the optimized sequence AZ3 encoding AADC, mir-183 and mir-122 target sequences, a bovine growth hormone (bGH) polyadenylation signal, a 3′ end AAV ITR sequence, and a backbone sequence.

### Embodiment 2

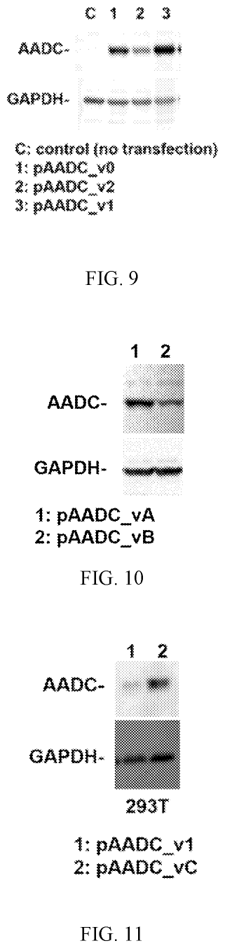

Using the plasmids pAADC_v0, pAADC_v1, and pAADC_v2 constructed in Embodiment 1, 293T cells (purchased from ATCC) were transfected with PEIpro® (from Polyplus) according to the reagent instructions. 3 μL of PEIpro® was used for 1 g of plasmid. Two days after transfection, the expression level of AADC was determined by immunoblotting (AADC antibody was purchased from Abcam, with catalog number ab3905), and the results are shown in FIG. 9.

The results show that pAADC_v0 (carrying the wild-type sequence encoding AADC), pAADC_v1 (carrying the optimized sequence AZ3) and pAADC_v2 (carrying the optimized sequence EF) vectors all expressed AADC protein in the cell. Compared with pAADC_v0, the expression efficiency of pAADC_v1 was significantly increased; the expression efficiency of pAADC_v2 did not increase significantly, but decreased to a certain extent. The results above show that the codon-optimized sequence AZ3 significantly improved the expression efficiency of AADC protein, but the sequence EF, which was also codon optimized, did not improve the expression efficiency. Furthermore, in addition to EF, the applicant also found a large number of optimized sequences that could not improve the expression efficiency during the screening process, confirming that the strategy of improving the expression efficiency by codon optimization will be affected by multiple factors such as protein type, protein length, secondary structure, and species type, and is unpredictable. Accordingly, the preferred sequence that can efficiently express AADC in the vector provided in the present application is AZ3.

### Embodiment 3

Using the plasmids pAADC_vA and pAADC_vB constructed in Embodiment 1, 293T cell line (purchased from ATCC) were transfected with PEIpro® (from Polyplus) according to the reagent instructions. 3 μL of PEIpro® was used for 1 g of plasmid. Two days after transfection, the expression level of AADC was determined by immunoblotting (AADC antibody was purchased from Abcam, with catalog number 3905). The results are shown in FIG. 10.

The results show that both vectors effectively expressed AADC protein in cells, and that the expression level of pAADC_vA (carrying the optimized sequence AZ3) is significantly higher than that of pAADC_vB (carrying the wild-type sequence encoding AADC). The results above show that the sequence AZ3 obtained by optimizing the AADC coding sequence in the present application can significantly improve the expression efficiency of AADC.

### Embodiment 4

Using the plasmids pAADC_v1 and pAADC_vC constructed in Embodiment 1, 293T cell line (purchased from ATCC) were transfected with PEIpro® (from Polyplus) according to the reagent instructions. 3 μL of PEIpro® was used for 1 g of plasmid. Two days after transfection, the expression level of AADC was determined by immunoblotting (AADC antibody was purchased from Abcam, with catalog number 3905). The results are shown in FIG. 11.

pAADC_v1 is a plasmid having mir-183 and mir-122 target sequences, and pAADC_vC does not have mir-183 and mir-122 target sequences. The results show that the addition of mir-183 and mir-122 target sequences can downregulate the expression level of AADC, indicating that the addition of miRNA target sequences to the vector can reduce the expression level of the gene of interest.

### Embodiment 5

A control scAAV vector comprising control miRNA target sequences was constructed in the same manner as in Embodiment 1. The construction method is briefly described as follows: The amplification primers of control miRNA (hsa-let-7a-1, has-miR-99a-5p, hsa-miR-100-5p, hsa-miR-802, hsa-miR-1200) were synthesized in the form of long primers. The primer sequences are shown in Table 1. The vector scAAV-MeP426-Mecp2-pA1-pA2 (SEQ ID NO: 21) was subjected to circular PCR and the miRNA target sequence was introduced. The amplified fragments above were cyclized by seamless cloning, and the ligation products were transformed into recombinase-deficient competent Escherichia. coli Stb13 cells (TransGen, CD521-01) for amplification to obtain a variety of control scAAV vectors comprising control miRNA target sequences.

TABLE 2
PCR primer sequences for control miRNA

| Name | Primer Name | Primer Sequence |
|---|---|---|
| hsa-let-7a-1 | let-7a-1-F | SEQ ID NO: 22 |
| | let-7a-1-R | SEQ ID NO: 23 |
| has-miR-99a-5p | 99a-5p-F | SEQ ID NO: 24 |
| | 99a-5p-R | SEQ ID NO: 25 |
| hsa-miR-100-5p | 100-5p-F | SEQ ID NO: 26 |
| | 100-5p-R | SEQ ID NO: 27 |
| hsa-miR-802 | 802-F | SEQ ID NO: 28 |
| | 802-R | SEQ ID NO: 29 |
| hsa-miR-1200 | 1200-F | SEQ ID NO: 30 |
| | 1200-R | SEQ ID NO: 31 |

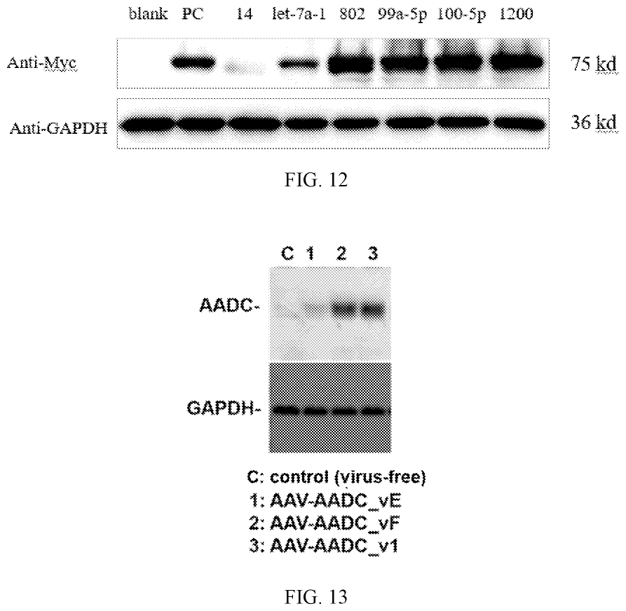

After the construction was completed, the control scAAV vector comprising control miRNA target sequences was transfected into Neuro-2a cells according to the method in Embodiment 2, and then the expression level of the protein of interest was determined by Western blot. The results are shown in FIG. 12. The different labels in the figure represent the use of different vectors for transfection:
Blank represents the untransfected blank group,
PC represents the control vector not comprising miR-183 and miR-122 target sequences (scAAV-MeP426-Mecp2-pA1-pA2, with the sequence of the exogenous nucleic acid fragment between its ITR sequences being as shown in SEQ ID NO: 32),
14 represents the vector comprising miR-183 and miR-122 target sequences (scAAV-MeP426-Mecp2-pA1-pA2-183-483-122-130, with the sequence of the exogenous nucleic acid fragment between its ITR sequences being as in shown in SEQ ID NO: 33),
Let-7a-1 represents the vector comprising the let-7a-1 target sequence (scAAV-MeP426-Mecp2-pA1-pA2-let-7a-1, with the sequence of the exogenous nucleic acid fragment between its ITR sequences being as shown in SEQ ID NO: 34),
802 represents the vector comprising the miR-802 target sequence (scAAV-MeP426-Mecp2-pA1-pA2-802, with the sequence of the exogenous nucleic acid fragment between its ITR sequences being as shown in SEQ ID NO: 37),
99a-5p represents the vector comprising the miR-99a-5p target sequence (scAAV-MeP426-Mecp2-pA1-pA2-99a-5p, with the sequence of the exogenous nucleic acid fragment between its ITR sequences being as shown in SEQ ID NO: 35),
100-5p represents the vector comprising the miR-100-5p target sequence (scAAV-MeP426-Mecp2-pA1-pA2-100-5p, with the sequence of the exogenous nucleic acid fragment between its ITR sequences being as shown in SEQ ID NO: 36), and
1200 represents the vector comprising the miR-1200 target sequence (scAAV-MeP426-Mecp2-pA1-pA2-1200, with the sequence of the exogenous nucleic acid fragment between its ITR sequences being as shown in SEQ ID NO: 38).

The results in FIG. 12 show that different combinations of miRNA target sequences have different effects on reducing the expression of the gene of interest.

### Embodiment 6

In the plasmids pAADC_v1, pAADC_vE, and pAADC_vF constructed in Embodiment 1, AAV-AADC_v1, AAV-AADC_vE, and AAV-AADC_vF were obtained by packaging the foresaid plasmids using AAV9. The plasmids used in the packaging step comprise: a capsid plasmid expressing AAV9 (Addgene 37825-AAV9.T), a helper plasmid (Genemedi P-HPO1), and a gene of interest plasmid used in each experiment (such as pAADC_v1, pAADC_vE, or pAADC_vF). The three plasmids were transfected in 293T cells (purchased from ATCC) at a molar ratio of 1:1:1, and a cell fluid with the virus was obtained. The specific transfection method is as follows:
1) Preparation of the complex: Prepare the DNA-PEI nucleic acid-transfection reagent complex according to the following system: For each well of cells, dilute 2 g of the plasmid of interest with 100 μL of serum-free culture medium and mix thoroughly to form a DNA dilution buffer. Immediately add 6 μL of PEIpro® transfection reagent (from Polyplus) to 100 μL of DNA dilution buffer and mix gently. Incubate at room temperature for 10-20 minutes to form a DNA-PEI nucleic acid transfection reagent complex.
2) Transfection of cells: Directly add 100 μL of DNA-PEI nucleic acid-PEI complex to the culture medium of 293T cells, shake the culture plate and gently mix. Culture in a 37° C., 5% CO2 incubator. The expression of the transgene can be determined as early as 48 hours after transfection.

The AAV-AADC_vE, AAV-AADC_vF and AAV-AADC_v1 obtained by the packaging above were used to infect the human 293T cell line. Two days later, the expression level of AADC was determined by immunoblotting (the AADC antibody was purchased from Abcam, with catalog number ab3905). The results are shown in FIG. 13.

The combination strategy of promoter, enhancer and regulatory sequence elements of pAADC_vE is derived from U.S. Ser. No. 10/898,585B2 (parts of other elements such as optimized sequence AZ3 are derived from the present application). By comparing the expression efficiency of pAADC_vE with that of pAADC_v1, it can be seen that the combination strategy of promoter, enhancer and regulatory sequence elements selected in the present application can further improve the expression efficiency of the optimized sequence AZ3, and is significantly better than the element combination strategy in the prior art U.S. Ser. No. 10/898,585B2. The results above show that although the element combination strategy in U.S. Ser. No. 10/898,585B2 can play a role in enhancing the expression efficiency of the gene of interest in the vector disclosed in the patent, it cannot play a role in the optimized sequence AZ3 provided in the present application.

### Embodiment 7

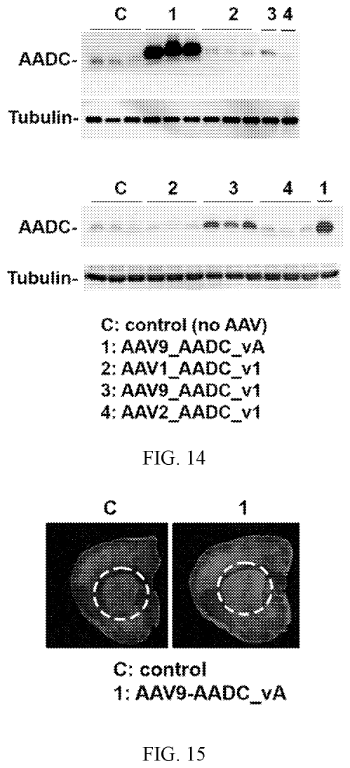

In the plasmids pAADC_vA and pAADC_v1 constructed in Embodiment 1, AAV9 was used to package the plasmid pAADC_vA to obtain AAV9-AADC_vA, and AAV1, AAV2, and AAV9 were used to package the plasmid pAADC_v1 to obtain AAV1-AADC_v1, AAV2-AADC_v1, and AAV9-AADC_v1. The packaging methods of AAV1, AAV2, and AAV9 are commonly used in the art (see Neurosci Res, 2015, 93: 144-57.doi: 10.1016/j.neures.2014.09.002). c57b1/6 mice were infected with an infection titer of 5E+10vg/mouse. After 21 days, the mouse brain was taken and the expression level of AADC was determined by immunoblotting (the AADC antibody was purchased from Abcam, with catalog number ab3905), and the results are shown in FIG. 14.

The results show that both AAV9-AADC_vA and AAV9-AADC_v1 effectively express the AADC protein in the mouse brain, and AAV9-AADC_vA has a higher expression efficiency. Since the expression activities of AAV-AADC_vF and AAV-AADC_v1 in cells in Embodiment 5 are comparable, and the expression activity of AAV9-AADC_vA is much higher than that of AAV-AADC_v1, AAV9-AADC_vA is the optimal strategy screened out.

Additionally, the left brain of the mouse was taken for frozen section immunostaining, and the results are shown in FIG. 15 with the striatum being framed. The result shows that AAV9-AADC_vA is well expressed in the striatum, the brain region of interest for AADC treatment.

### Embodiment 8

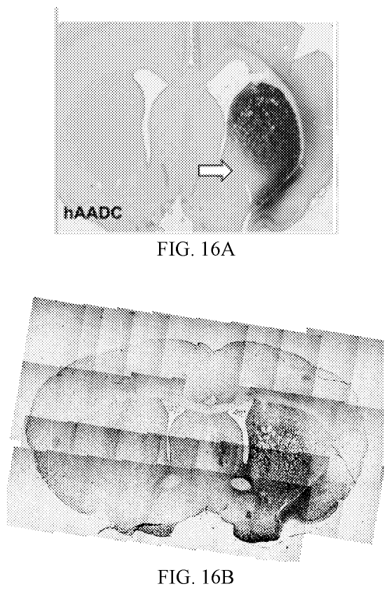

The AAV9-AADC_vA obtained in Embodiment 7 was injected into the right striatum of SD rats by microinjection at a dose of 1.5E+1 vg per animal. After 2-4 weeks, the rat brain was taken and fixed with 4% PFA, and whole brain frozen sections were made. The brain sections were stained by DAB staining (the AADC antibody was purchased from Abcam, with catalog number ab3905; DAB color development kit was purchased from Beyotime, with catalog number P0202). The distribution map of AADC in the brain was obtained by taking pictures with a microscope and then assembling them, as shown in FIG. 16B.

The expression location of other AADC expression vector AAV2-hAADC in the prior art (see PLoS One, 2015, 10 (4): e0122708.doi: 10.1371/journal.pone.0122708) is more limited to the area around the injection site (see FIG. 3A in PLoS One, 2015, 10 (4): e0122708., and FIG. 16A in the present application). Compared with AAV2-hAADC, the vector AAV9-AADC_vA provided in the present application is expressed in a wider range in the same part of rats, and can diffuse out of the injection site and diffusely distribute throughout the striatum (FIG. 16B). As shown in the figure, the white arrow in FIG. 16A is the region where AAV2-AADC has no AADC expression in the literature, but AAV9-AADC_vA provided in the present application is expressed in this region. Since the volume of human striatum is much larger than that of a rat, the better diffusion of AAV9-AADC_vA indicates that it has a better therapeutic efficacy.

### Embodiment 9

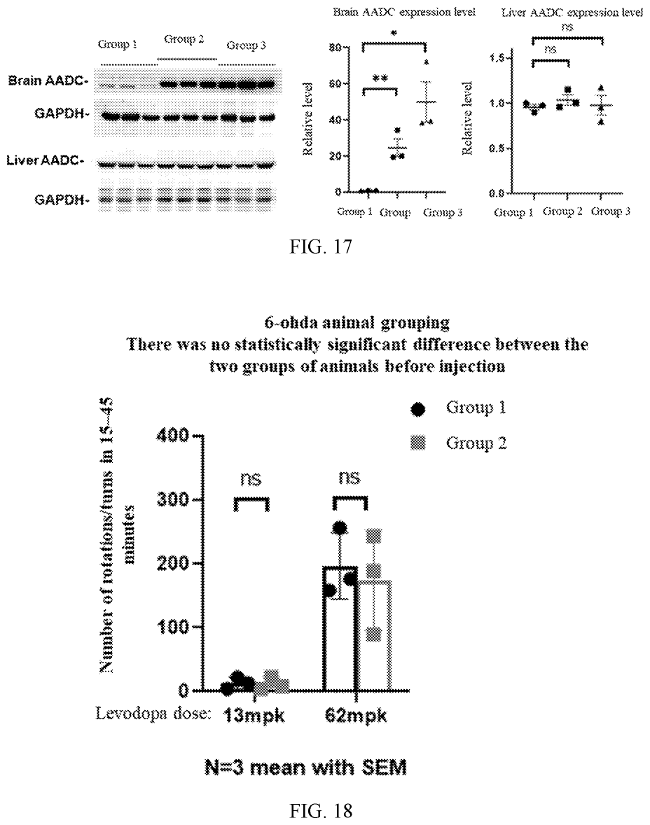

The AAV9-AADC_vE obtained in Embodiment 6 or the AAV9-AADC_vA obtained in Embodiment 7 was injected into the right striatum of c57bl/6 mice by microinjection at a dose of 3.4E+10vg per animal. After 21 days, the mouse brain and liver were taken and the expression level of AADC was determined by immunoblotting (the AADC antibody was purchased from Abcam, with catalog number ab3905), and the results are shown in FIG. 17. Group 1 is the test results of 3 mice injected with empty capsids (the virus does not carry the AADC gene); Group 2 is the test results of 3 mice injected with the virus AAV9-AADC_vE; Group 3 is the test results of 3 mice injected with the virus AAV9-AADC_vA. Additionally, the grayscale values of the immunoblot bands were read using the software Clinx Image Analysis, and statistical analysis was performed using GraphPad Prism. The t-test was used to perform a significance analysis of the differences between the two groups of data marked in the figure, where * and ** represent statistically significant difference, and ns represents no statistically significant difference.

The results in FIG. 17 show that animals injected with AAV9-AADC_vE and AAV9-AADC_vA were found to have increased expression in brain tissues, proving that AADC can be expressed in mouse brain tissues; additionally, the AADC expression level of AAV9-AADC_vA was significantly higher than that of the control vector AAV9-AADC_vE. It is proved that the expression efficiency of AAV9-AADC_vA in the brain is better than that of AAV9-AADC_vE. The results above once again verified the results of Embodiment 6, confirming that although the element combination strategy in U.S. Ser. No. 10/898,585B2 can play a role in enhancing the expression efficiency of the gene of interest in the vector disclosed in the patent, it cannot play a role in the optimized sequence AZ3 provided in the present application.

The results of FIG. 17 also show that the expression level of AADC in the liver of animals injected with AAV9-AADC_vE and AAV9-AADC_vA did not increase, indicating that the vectors to which the mir-183 and mir-122 target sequences were added would not lead to increased expression of AADC in the liver, and thus the liver toxicity was relatively small. All vectors provided in the present application did not cause obvious dorsal root ganglion lesions (with the result being not shown), confirming that the addition of miRNA target sequences (such as mir-183 and mir-122 target sequences) to the vectors can also effectively reduce the dorsal root ganglion toxicity caused by AAV drug administration. The results above confirm that the vectors in the present application can solve the toxicity problems caused by traditional AAV vectors and are a more preferred treatment option.

### Embodiment 10

After establishing a Parkinson's disease model by unilateral intraventricular injection of 6-ohda in the brains of SD rats, levo-dopamine was injected intraperitoneally, and rotation-positive animals were selected for the test. The positive animals were randomly divided into two groups for behavioral testing: different doses of levo-dopamine (13 mpk, 62 mpk) were injected intraperitoneally into the animals every day, and the number of rotations of the animals from 15 minutes to 45 minutes after the injection was counted. The results are shown in FIG. 18. The two groups of data marked in the figure were statistically analyzed using t-test, and ns represents no statistically significant difference. The results in FIG. 17 show that there was no statistically significant difference in behavior between the two groups of animals before treatment with AAV drugs.

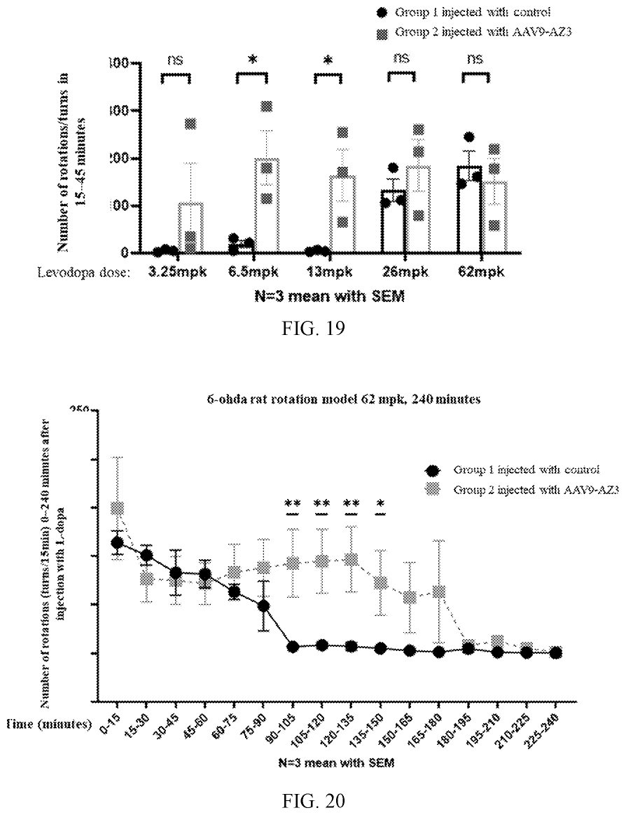

Subsequently, AAV9-AADC_vA obtained in Embodiment 7 was injected into the right striatum of the two groups of animals by microinjection at doses of 1.5E+11vg, 2.76E+10vg, 9.21E+9vg, and 3.07E+9vg per animal. Behavioral testing began in the third week after the injection: different doses of levo-dopamine (3.25 mpk, 6.5 mpk, 13 mpk, 26 mpk, and 62 mpk) were injected intraperitoneally into the animals daily, and the number of rotations from 15 minutes to 45 minutes after the injection into the animals was counted, and the results are shown in FIG. 19. In the figure, Group 1 is the test results of rats injected with empty capsids (the virus does not carry the AADC gene); Group 2 is the test results of rats injected with the virus AAV9-AADC_vA. The two groups of data marked in the figure were statistically analyzed using t-test, * and ** represent statistically significant difference, and ns represents no statistically significant difference.

The results in FIG. 19 show that rats injected with AAV9-AADC_vA can induce obvious rotation at lower levodopa doses (e.g., 6.5 mpk and 13 mpk), proving that AAV9-AADC_vA can reduce the dosage of levodopa while achieving the effect of high-dose levodopa.

Additionally, the same dose of levodopa (62 mpk) was injected intraperitoneally into the animals, and the number of rotations of the animals after injection was counted. The results are shown in FIG. 20. Group 1 is the test results of rats injected with empty capsids (the virus does not carry the AADC gene); Group 2 is the test results of rats injected with the virus AAV9-AADC_vA. The two groups of data marked in the figure were statistically analyzed using Fisher's LSD test, and * and ** represent statistically significant difference. The results in FIG. 20 confirm that the rotation time of animals injected with AAV9-AADC_vA is significantly longer.

The results of FIGS. 18-20 above indicate that the use of AAV9-AADC_vA in Parkinson's model animals can significantly reduce the dosage of levodopa and can prolong the duration of action of the drug levodopa.

### Embodiment 11

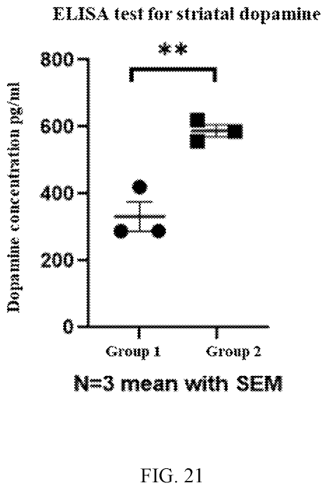

The rats injected with empty capsids (Group 1) and the rats injected with the virus AAV9-AADC_vA (Group 2) in Embodiment 10 were killed 1 hour after the intraperitoneal injection of levodopa, the striatum on the side injected with 6-ohda was immediately removed, and the striatal dopamine content was determined and statistically analyzed using the ELISA method (Elabscience, with catalog number E-EL-0046), with the results being shown in FIG. 21. The two groups of data marked in the figure were determined using t-test, and ** represents statistically significant difference.

The results in FIG. 21 show that the brain tissue of rats injected with AAV9-AADC_vA comprises more dopamine, that is, AAV9-AADC_vA can improve the efficiency of converting levodopa into dopamine at the injection site. Accordingly, expressing AADC in the striatum to increase the conversion of levodopa into dopamine is the mechanism of action of the AAV vector provided in the present application for treating Parkinson's disease.

It is to be stated that what have been discussed above are only preferred embodiments of the present application, not for limiting the present application. For those skilled in the art, the present application may have various changes and variations. While particular embodiments have been described, alternatives, modifications, variations, improvements, and substantial equivalents of the embodiments above may exist or be presently unforeseen to the applicants or others skilled in the art. Accordingly, the appended claims as filed and as they may be amended are intended to embrace all such alternatives, modifications, variations, improvements, and substantial equivalents. Importantly, with the evolution of the technology, many elements described herein may be replaced by equivalent elements appearing after the present application.

1. A nucleic acid fragment, comprising a nucleotide sequence encoding aromatic L-amino acid decarboxylase (AADC), wherein the nucleotide sequence encoding AADC comprises a nucleotide sequence having at least 80%, 85%, 90%, 95%, 98%, 99%, or 100% identity to the nucleotide sequence shown in SEQ ID NO: 3.

2. The nucleic acid fragment according to claim 1, wherein the nucleotide sequence encoding AADC comprises the nucleotide sequence shown in SEQ ID NO: 3.

3. An expression vector, wherein the expression vector comprises the nucleic acid fragment according to claim 1.

4. The expression vector according to claim 3, wherein the expression vector is a plasmid vector or a viral vector.

5. The expression vector according to claim 4, wherein the viral vector comprises an adeno-associated virus (AAV) vector.

6. The expression vector according to claim 5, wherein the expression vector further comprises a 5′ end AAV inverted terminal repeat (ITR) sequence and a 3′ end AAV ITR sequence.

7. The expression vector according to claim 6, wherein the 5′ end AAV ITR sequence comprises the nucleotide sequence shown in SEQ ID NO: 12 or SEQ ID NO: 14, and the 3′ end AAV ITR sequence comprises the nucleotide sequence shown in SEQ ID NO: 13 or SEQ ID NO: 39.

8. The expression vector according to claim 3, further comprising miRNA target sequences, wherein:
the miRNA target sequences can reduce the expression efficiency of the nucleotide sequence encoding AADC in liver tissues; and/or
the miRNA target sequences can reduce the expression efficiency of the nucleotide sequence encoding AADC in dorsal root ganglion tissues.

9. The expression vector according to claim 8, wherein the miRNA target sequences comprise one or both of the miR-183 target sequence and the miR-122 target sequence.

10. The expression vector according to claim 9, wherein the number of miRNA target sequences is 1, 2, 3, 4, 5, 6, 7, or 8.

11. The expression vector according to claim 9, wherein the miRNA target sequences comprise 2 miR-183 target sequences and 2 miR-122 target sequences.

12. The expression vector according to claim 3, further comprising:
a promoter, wherein the promoter comprises a chicken beta actin (CBA) promoter, a cytomegalovirus (CMV) promoter, and a human SYN1 gene promoter;
an enhancer, wherein the enhancer comprises a CMV enhancer; and/or
a polyadenylation signal, wherein the polyadenylation signal comprises a simian vacuolating virus 40 (SV40) polyadenylation signal or a bovine growth hormone (bGH) polyadenylation signal.

13-16. (canceled)

17. The expression vector according to claim 3, comprising, in sequence from the 5′ end to the 3′ end:
a) a 5′ end AAV ITR sequence;
b) optionally, an enhancer;
c) a promoter;
d) the nucleic acid fragment according to claim 1 or 2;
e) miRNA target sequences;
f) a polyadenylation signal; and
g) a 3′ end AAV ITR sequence.

18. A recombinant adeno-associated virus (rAAV), comprising the expression vector according to claim 3.

19. The recombinant adeno-associated virus according to claim 18, wherein the serotype of the rAAV comprises AAV1, AAV2, AAV3, AAV4, AAV5, AAV6, AAV7, AAV8, AAV9, AAV9.47, AAV9 (hu14), AAV10, AAV11, AAV12, AAV13, AAVrh8, AAVrh10, AAV-DJ, or AAV-DJ8.

20. The recombinant adeno-associated virus according to claim 19, wherein the serotype of the rAAV comprises AAV1, AAV2, or AAV9.

21. A pharmaceutical composition, comprising the expression vector according to claim 3, and pharmaceutically, physiologically, or nutritionally acceptable excipients.

22. The pharmaceutical composition according to claim 21, wherein the pharmaceutical composition is administered by intravenous injection or suboccipital injection.

23-25. (canceled)

26. A method for preventing, alleviating, or treating a neurological disease, comprising administering to a subject in need thereof a therapeutically effective amount of the expression vector according to claim 3.

27. The method according to claim 26, wherein the neurological disease is a neurological disease caused by a deficiency or reduced activity of aromatic L-amino acid decarboxylase.

28. The method according to claim 27, wherein the neurological disease comprises Parkinson's disease and aromatic L-amino acid decarboxylase deficiency.

29. The expression vector according to claim 9, wherein the miRNA target sequences comprise one or both of the nucleotide sequences shown in SEQ ID NO: 5 and SEQ ID NO: 6.

30. The expression vector according to claim 9, wherein the number of miRNA target sequences is 4.

31. The recombinant adeno-associated virus according to claim 20, wherein the serotype is AAV9.


SEQUENCE LISTING (39 sequences)

SEQ ID NO: 1 - DNA, 1443 bp, synthetic construct
  source 1..1443: mol_type = other DNA
atgaacgcaagtgaattccgaaggagagggaaggagatggtggattacatggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtag

SEQ ID NO: 2 - protein, 480 aa, synthetic construct
MNASEFRRRGKEMVDYMANYMEGIEGRQVYPDVEPGYLRPLIPAAAPQEPDTFEDIINDVEKIIMPGVTHWHSPYFFAYFPTASSYPAMLADMLCGAIGCIGFSWAASPACTELETVMMDWLGKMLELPKAFLNEKAGEGGGVIQGSASEATLVALLAARTKVIHRLQAASPELTQAAIMEKLVAYSSDQAHSSVERAGLIGGVKLKAIPSDGNFAMRASALQEALERDKAAGLIPFFMVATLGTTTCCSFDNLLEVGPICNKEDIWLHVDAAYAGSAFICPEFRHLLNGVEFADSFNFNPHKWLLVNFDCSAMWVKKRTDLTGAFRLDPTYLKHSHQDSGLITDYRHWQIPLGRRFRSLKMWFVFRMYGVKGLQAYIRKHVQLSHEFESLVRQDPRFEICVEVILGLVCFRLKGSNKVNEALLQRINSAKKIHLVPCHLRDKFVLRFAICSRTVESAHVQRAWEHIKELAADVLRAERE

SEQ ID NO: 3 - DNA, 1443 bp, synthetic construct
  source 1..1443: mol_type = other DNA
atgaacgccagcgagtttcggcggagaggcaaagaaatggtggactacatggccaactacatggaaggcatcgagggcagacaggtgtaccccgatgtggaacctggctacctgaggcctctgattcctgccgctgctccccaagagcctgacaccttcgaggacatcatcaacgacgtggaaaagatcatcatgcccggcgtgacccactggcacagcccctactttttcgcctactttcccaccgccagcagctaccctgctatgctggctgatatgctgtgtggcgccatcggctgcatcggcttttcttgggctgcctctcctgcctgcaccgagctggaaaccgtgatgatggattggctgggcaagatgctggaactgcccaaggccttcctgaacgagaaagctggcgaaggcggcggagtgattcagggatctgcctctgaagccacactggtggctctgctggccgccagaacaaaagtgatccacagactgcaggccgcctctccagaactgacacaggccgccatcatggaaaagctggtggcctacagcagcgatcaggcccacagctctgtggaaagagccggactgattggcggcgtgaagctgaaggccattcctagcgacggcaacttcgccatgagagcctctgctctgcaagaggccctggaaagagataaggccgctggactgatccccttcttcatggtggctaccctgggcaccaccacctgttgcagcttcgacaacctgctggaagtgggccccatctgcaacaaagaggacatctggctgcacgtggacgccgcctatgccggctctgcctttatctgccccgagttcagacatctgctgaacggcgtggaattcgccgacagcttcaacttcaaccctcacaagtggctgctggtcaacttcgactgcagcgctatgtgggtcaagaagcggaccgatctgaccggcgccttcagactggatcccacctacctgaagcactcccaccaggatagcggcctgatcaccgactaccggcactggcagattcctctgggcagaagatttcggagcctgaagatgtggttcgtgttccggatgtatggcgtgaagggcctccaggcctacatcagaaaacatgtgcagctgagccacgagttcgagagccttgtcagacaggaccccagattcgagatctgcgtggaagtgatcctgggcctcgtgtgctttagactgaagggcagcaacaaagtgaacgaggccctgctgcagcggatcaacagcgccaaaaagatccacctggtgccttgccacctgagagacaagttcgtgctgagattcgccatctgcagccggacagtggaaagcgcccatgtgcagagagcctgggagcacatcaaagaactggccgccgatgtgctgagggccgagagagaatga

SEQ ID NO: 4 - DNA, 1443 bp, synthetic construct
  source 1..1443: mol_type = other DNA
atgaatgcctcagaattccggagaagaggcaaagagatggtggattacatggccaactacatggaggggatagagggacgacaggtatatccggacgtagagcctggatatctcaggcctctgatcccagctgccgctcctcaagagccagatacctttgaggacattatcaatgacgtcgagaagattatcatgccaggggtgacccattggcacagtccgtacttcttcgcatatttcccgactgcgagcagctaccctgccatgctggctgatatgctctgtggtgcaattgggtgcatcggatttagctgggcagcaagcccagcgtgtacagagctggagactgtgatgatggactggttggggaaaatgctcgaactgcccaaggccttcctcaacgagaaagccggagaaggcggaggggtgattcagggttcagcctcagaggctacactggtagctctgctggccgcaagaactaaggtcatacaccgccttcaggctgctagtcctgaactgacacaagccgccatcatggagaagcttgttgcctattcttccgatcaggcacattcttccgttgaaagagcagggctgataggtggagtgaagctgaaggcaattcccagtgatggcaatttcgctatgcgtgcctctgcgctccaggaagcgttggaacgggacaaagcagctggactgatcccattcttcatggtagcgactctgggtacaaccacgtgctgcagtttcgacaaccttctggaggttggccccatttgcaacaaggaggacatttggttgcacgttgacgcagcatatgctggttctgcctttatctgccccgaatttcggcacctgttgaatggcgtggaatttgccgacagcttcaacttcaatccccacaaatggctgctggtgaactttgactgttctgccatgtgggttaagaaacgaaccgaccttacaggcgcttttcgcctggatccaacgtacctgaagcactcccaccaggattccggcctgataaccgattacagacattggcagattcctttgggacgaagatttcggtccctgaagatgtggtttgtgtttaggatgtatggcgtgaaagggttgcaggcctacatacggaaacatgtgcagctgtctcacgagtttgaatccctggtcaggcaggatccccgtttcgaaatctgcgtggaagtgattcttggcctcgtgtgcttccggctgaaaggcagcaataaggtcaatgaggctctcctccaacgcatcaacagcgccaagaagatccatctcgtgccctgtcatctgagggacaaattcgtccttcgctttgccatctgttcaaggaccgttgagagcgctcatgtccaaagggcctgggaacacatcaaggagctggccgctgatgtcctgcgcgcagaacgcgagtga

SEQ ID NO: 5 - DNA, 22 bp, synthetic construct
  source 1..22: mol_type = other DNA
caaacaccattgtcacactcca

SEQ ID NO: 6 - DNA, 22 bp, synthetic construct
  source 1..22: mol_type = other DNA
agtgaattctaccagtgccata

SEQ ID NO: 7 - DNA, 204 bp, synthetic construct
  source 1..204: mol_type = other DNA
gtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagct

SEQ ID NO: 8 - DNA, 380 bp, synthetic construct
  source 1..380: mol_type = other DNA
gagattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 9 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 10 - DNA, 1113 bp, synthetic construct
  source 1..1113: mol_type = other DNA
gtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 11 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 12 - DNA, 106 bp, synthetic construct
  source 1..106: mol_type = other DNA
ctgcgcgctcgctcgctcactgaggccgcccgggcaaagcccgggcgtcgggcgacctttggtcgcccggcctcagtgagcgagcgagcgcgcagagagggagtgg

SEQ ID NO: 13 - DNA, 143 bp, synthetic construct
  source 1..143: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 14 - DNA, 139 bp, synthetic construct
  source 1..139: mol_type = other DNA
tgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 15 - DNA, 3632 bp, synthetic construct
  source 1..3632: mol_type = other DNA
cccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagattatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgcc

SEQ ID NO: 16 - DNA, 17 bp, synthetic construct
  source 1..17: mol_type = other DNA
taggtcaatgagggatg

SEQ ID NO: 17 - DNA, 17 bp, synthetic construct
  source 1..17: mol_type = other DNA
acttgggagtcctatga

SEQ ID NO: 18 - DNA, 17 bp, synthetic construct
  source 1..17: mol_type = other DNA
agactgatgtaaactag

SEQ ID NO: 19 - DNA, 448 bp, synthetic construct
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 20 - DNA, 88 bp, synthetic construct
  source 1..88: mol_type = other DNA
caaacaccattgtcacactccaagtgaattctaccagtgccatacaaacaccattgtcacactccaagtgaattctaccagtgccata

SEQ ID NO: 21 - DNA, 2205 bp, synthetic construct
  source 1..2205: mol_type = other DNA
ataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagcgggagctccggcagttctggagaacaaaaactcatctcagaagaggatctgtgaacaagaataaaggcagctgttgtctcttctccttatgggtagggctctgacaaagcttcccatatcaccaggactgttaatatatttaaaaacaaaaggcaatttattaaggaaatttgtaccatttcagtaaacctgtctgaaccggagagaccgagagagggtctcagttttataaagagtttgccttataaatttacataaaaatgtccgtttgtgtcttttgttg

SEQ ID NO: 22 - DNA, 53 bp, synthetic construct
  source 1..53: mol_type = other DNA
aactatacaacctactacctcagttttataaagagtttgccttataaatttac

SEQ ID NO: 23 - DNA, 49 bp, synthetic construct
  source 1..49: mol_type = other DNA
tgaggtagtaggttgtatagttccggttcagacaggtttactgaaatgg

SEQ ID NO: 24 - DNA, 53 bp, synthetic construct
  source 1..53: mol_type = other DNA
cacaagatcggatctacgggttgttttataaagagtttgccttataaatttac

SEQ ID NO: 25 - DNA, 49 bp, synthetic construct
  source 1..49: mol_type = other DNA
aacccgtagatccgatcttgtgccggttcagacaggtttactgaaatgg

SEQ ID NO: 26 - DNA, 53 bp, synthetic construct
  source 1..53: mol_type = other DNA
cacaagttcggatctacgggttgttttataaagagtttgccttataaatttac

SEQ ID NO: 27 - DNA, 49 bp, synthetic construct
  source 1..49: mol_type = other DNA
aacccgtagatccgaacttgtgccggttcagacaggtttactgaaatgg

SEQ ID NO: 28 - DNA, 54 bp, synthetic construct
  source 1..54: mol_type = other DNA
acaaggatgaatctttgttactggttttataaagagtttgccttataaatttac

SEQ ID NO: 29 - DNA, 50 bp, synthetic construct
  source 1..50: mol_type = other DNA
cagtaacaaagattcatccttgtccggttcagacaggtttactgaaatgg

SEQ ID NO: 30 - DNA, 53 bp, synthetic construct
  source 1..53: mol_type = other DNA
gaggctcagaatggctcaggaggttttataaagagtttgccttataaatttac

SEQ ID NO: 31 - DNA, 49 bp, synthetic construct
  source 1..49: mol_type = other DNA
ctcctgagccattctgagcctcccggttcagacaggtttactgaaatgg

SEQ ID NO: 32 - DNA, 2205 bp, synthetic construct; identical to SEQ ID NO: 21
  source 1..2205: mol_type = other DNA

SEQ ID NO: 33 - DNA, 2303 bp, synthetic construct
  source 1..2303: mol_type = other DNA
ataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagcgggagctccggcagttctggagaacaaaaactcatctcagaagaggatctgtgaacaagaataaaggcagctgttgtctcttctccttatgggtagggctctgacaaagcttcccatatcaccaggactgttaatatatttaaaaacaaaaggcaatttattaaggaaatttgtaccagtgaattctaccagtgccatagtacctgtatacgtttcaaaaactcccttctttcctcccgtctacaccccccccccactgaatccaaacaccattgtcacactccacctgtaacctatttattaatgcccttttaacattgcactatataaagagtttgccttataaatttacataaaaatgtccgtttgtgtcttttgttg

SEQ ID NO: 34 - DNA, 4285 bp, synthetic construct
  source 1..4285: mol_type = other DNA
tcgcgcgtttcggtgatgacggtgaaaacctctgacacatgcagctcccggacgtcattgtcgatgctagcctgcgcgctcgctcgctcactgaggccgcccgggcaaagcccgggcgtcgggcgacctttggtcgcccggcctcagtgagcgagcgagcgcgcagagagggagtggataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagcgggagctccggcagttctggagaacaaaaactcatctcagaagaggatctgtgaacaagaataaaggcagctgttgtctcttctccttatgggtagggctctgacaaagcttcccatatcaccaggactgttaatatatttaaaaacaaaaggcaatttattaaggaaatttgtaccatttcagtaaacctgtctgaaccggaactatacaacctactacctcagttttataaagagtttgccttataaatttacataaaaatgtccgtttgtgtcttttgttgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccatcattgtcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 35 - DNA, 4285 bp, synthetic construct
  source 1..4285: mol_type = other DNA
tcgcgcgtttcggtgatgacggtgaaaacctctgacacatgcagctcccggacgtcattgtcgatgctagcctgcgcgctcgctcgctcactgaggccgcccgggcaaagcccgggcgtcgggcgacctttggtcgcccggcctcagtgagcgagcgagcgcgcagagagggagtggataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagcgggagctccggcagttctggagaacaaaaactcatctcagaagaggatctgtgaacaagaataaaggcagctgttgtctcttctccttatgggtagggctctgacaaagcttcccatatcaccaggactgttaatatatttaaaaacaaaaggcaatttattaaggaaatttgtaccatttcagtaaacctgtctgaaccggcacaagatcggatctacgggttgttttataaagagtttgccttataaatttacataaaaatgtccgtttgtgtcttttgttgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccatcattgtcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 36 - DNA, 4285 bp, synthetic construct
  source 1..4285: mol_type = other DNA
tcgcgcgtttcggtgatgacggtgaaaacctctgacacatgcagctcccggacgtcattgtcgatgctagcctgcgcgctcgctcgctcactgaggccgcccgggcaaagcccgggcgtcgggcgacctttggtcgcccggcctcagtgagcgagcgagcgcgcagagagggagtggataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagcgggagctccggcagttctggagaacaaaaactcatctcagaagaggatctgtgaacaagaataaaggcagctgttgtctcttctccttatgggtagggctctgacaaagcttcccatatcaccaggactgttaatatatttaaaaacaaaaggcaatttattaaggaaatttgtaccatttcagtaaacctgtctgaaccggcacaagttcggatctacgggttgttttataaagagtttgccttataaatttacataaaaatgtccgtttgtgtcttttgttgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccatcattgtcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 37 - DNA, 4286 bp, synthetic construct
  source 1..4286: mol_type = other DNA
tcgcgcgtttcggtgatgacggtgaaaacctctgacacatgcagctcccggacgtcattgtcgatgctagcctgcgcgctcgctcgctcactgaggccgcccgggcaaagcccgggcgtcgggcgacctttggtcgcccggcctcagtgagcgagcgagcgcgcagagagggagtggataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagcgggagctccggcagttctggagaacaaaaactcatctcagaagaggatctgtgaacaagaataaaggcagctgttgtctcttctccttatgggtagggctctgacaaagcttcccatatcaccaggactgttaatatatttaaaaacaaaaggcaatttattaaggaaatttgtaccatttcagtaaacctgtctgaaccggacaaggatgaatctttgttactggttttataaagagtttgccttataaatttacataaaaatgtccgtttgtgtcttttgttgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccatcattgtcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 38 - DNA, 4285 bp, synthetic construct
  source 1..4285: mol_type = other DNA
tcgcgcgtttcggtgatgacggtgaaaacctctgacacatgcagctcccggacgtcattgtcgatgctagcctgcgcgctcgctcgctcactgaggccgcccgggcaaagcccgggcgtcgggcgacctttggtcgcccggcctcagtgagcgagcgagcgcgcagagagggagtggataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagcgggagctccggcagttctggagaacaaaaactcatctcagaagaggatctgtgaacaagaataaaggcagctgttgtctcttctccttatgggtagggctctgacaaagcttcccatatcaccaggactgttaatatatttaaaaacaaaaggcaatttattaaggaaatttgtaccatttcagtaaacctgtctgaaccgggaggctcagaatggctcaggaggttttataaagagtttgccttataaatttacataaaaatgtccgtttgtgtcttttgttgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccatcattgtcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 39 - DNA, 139 bp, synthetic construct
  source 1..139: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgca